(19) United States
(12) Patent Application Publication
(10) Pub. No.: US 2006/0035275 A1
(43) Pub. Date: Feb. 16, 2006

(54) Nucleic acid diagnostic reagents and methods for detecting nucleic acids, polynucleotides and oligonucleotides

(75) Inventors: David C. Ward, Old Lyme, CT (US); Ronald Breaker, Guilford, CT (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Yale University, New Haven, CT (US)

(21) Appl. No.: 11/264,859

(22) Filed: Nov. 2, 2005

Related U.S. Application Data
(63) Continuation of application No. 10/320,191, filed on Dec. 16, 2002, now abandoned.
(60) Provisional application No. 60/341,658, filed on Dec. 14, 2001.

(51) Int. Cl. C12Q 1/68; C12P 19/34

(52) U.S. Cl. 435/6; 435/91.2

(57) ABSTRACT

Methods for generating nucleic acid reagents useful for detecting nucleic acids, polynucleotides, and oligonucleotides are disclosed. Selection techniques, enzymatic nucleic acid molecules, allozymes (allosteric nucleic acid sensor molecules), ribozymes, and DNAzymes used as diagnostic reagents and tools are described.

This application is a continuation of U.S. patent application Ser. No. 10/320,191, filed Dec. 16, 2002, which claims the benefit of U.S. Provisional Application No. 60/341,658, filed Dec. 14, 2001. The instant application claims the benefit of all the listed applications, which are hereby incorporated by reference herein in their entireties, including the drawings.

### FIELD OF THE INVENTION

The present invention relates to nucleic acid-based reagents for detecting nucleic acids, including polynucleotides and oligonucleotides such as RNA and DNA, in a system. The invention is further related to methods of generating nucleic acid diagnostic reagents that are specific for one or more target nucleic acids, such as polynucleotides, and/or oligonucleotides. The methods and reagents of the invention can be used to identify the presence of nucleic acids, such as polynucleotides and/or oligonucleotides, in a system which are indicative of a particular genotype and/or phenotype, for example, a disease state, infection, or related disorder within an organism.

### BACKGROUND OF THE INVENTION

The following is a brief description of diagnostic and sensor-based applications for nucleic acids. This summary is provided only for understanding of the invention that follows. This summary is not an admission that the work described below is prior art to the claimed invention.

The detection of biomolecules, for example nucleic acids, can be highly beneficial in the diagnosis of diseases or medical disorders. By determining the presence of a specific nucleic acid sequence, investigators can confirm the presence of a virus, bacterium, genetic mutation, and/or other condition that relates to a disease. Assays for nucleic acid sequences can range from simple methods for detection, such as northern blot hybridization using a radiolabeled or fluorescent probe to detect the presence of a nucleic acid molecule, to the use of polymerase chain reaction (PCR) to amplify a small quantity of a specific nucleic acid to the point at which it can be used for detection of the sequence by hybridization techniques. The polymerase chain reaction uses DNA polymerases to logarithmically amplify the desired sequence using prefabricated primers to locate specific sequences (see for example U.S. Pat. No. 4,683,195 and U.S. Pat. No. 4,683,202). Nucleotide probes can be labeled using dyes, fluorescent, chemiluminescent, radioactive, or enzymatic labels which are commercially available. These probes can be used to detect by hybridization, the expression of a gene or related sequences in cells or tissue samples in which the gene is a normal component, as well as to screen sera or tissue samples from humans suspected of having a disorder arising from infection with an organism, or to detect novel or altered genes as might be found in tumorigenic cells. Nucleic acid primers can also be prepared which, with reverse transcriptase or DNA polymerase and PCR, can be used for detection of nucleic acid molecules that are present in very small amounts in tissues or fluids. PCR has several disadvantages, for example requiring a high degree of technical competence for reliability, high reagent costs, and sensitivity to contamination resulting in false positives.

Several groups have completed draft sequences of the entire human genome. To capitalize on this information, an effort to correlate changes in specific mRNA levels with different disease states has been initiated. The synergy of these efforts has been highly successful and currently information relating specific changes in gene expression to disease states is being developed. The further development of information is essential for the generation of treatment outcomes data that link patient and disease characteristics with future treatment events. Therefore, a clear need exists for molecular tools that can generate disease specific genomes or diagnostic molecular profiles that correlate individual cellular and molecular events with disease outcomes profiles. These profiles can then be used to rationally drive treatment policy decisions resulting in better patient care and reductions in health care spending.

A class of enzymes which can be utilized for diagnostic and sensor purposes are enzymatic nucleic acid molecules (Kuwabara et al., 2000, Curr. Opin. Chem. Bio., 4, 669; Porta et al., 1995, Biochemistry, 13, 161; Soukup et al., 1999, TIBTECH, 17, 469, Marshall et al., 1999, Nature Struc Biol., 6, 992). The enzymatic nature of an enzymatic nucleic acid sensor molecule can be advantageous over other sensor technologies, since the concentration of analyte necessary to generate a detectable response is generally lower than that required with other sensor systems which may require amplification steps. This advantage reflects the ability of the enzymatic nucleic acid molecule to act enzymatically. Thus, a specific enzymatic nucleic acid molecule is able to amplify a given signal in response to a single recognition event. Such enzymatic nucleic acid-based sensor molecules are often referred to in the art as allozymes, allosteric ribozymes, or allosteric DNAzymes.

In addition, the enzymatic nucleic acid molecule is a highly specific sensor molecule that can be engineered to respond to a variety of different signaling events. The use of in vitro selection techniques can be applied to the selection of new enzymatic nucleic acid molecules that are capable of allosteric modulation. Previous work in this area has focused on combining known aptamer and enzymatic nucleic acid molecule sequences (Breaker, International PCT Publication No. WO 98/2714). Later work has revealed bridge sequences that connect the receptor and enzymatic sequence domains together. These bridging sequences function such that binding of a ligand to the receptor domain triggers a conformational change within the bridge, thus modulating phosphodiester cleavage activity of the adjoining enzymatic sequence (Breaker, International PCT Publication No. WO 00/26226).

George et al., U.S. Pat. Nos. 5,834,186 and 5,741,679, describe regulatable RNA molecules whose activity is altered in the presence of a ligand.

Shih et al., U.S. Pat. No. 5,589,332, describe a method for the use of ribozymes to detect macromolecules such as proteins and nucleic acid.

Nathan et al., U.S. Pat. No. 5,871,914, describe a method for detecting the presence of an assayed nucleic acid based on a two component ribozyme system containing a detection ensemble and an RNA amplification ensemble.

Nathan and Ellington, International PCT publication No. WO 00/24931, describe the detection of an analyte by a ribozyme sequence which converts a nucleic acid substrate to a ribozyme product in the presence of the analyte. The ribozyme product is then amplified by PCR.

Sullenger et al., International PCT publication No. WO 99/29842, describe nucleic acid mediated RNA tagging and RNA revision.

Usman et al., International PCT Publication No. WO 01/66721, describes nucleic acid sensor molecules.

Nathan et al., International PCT Publication No. WO 98/08974, describes specific cofactor-dependent ribozyme constructs.

### SUMMARY OF THE INVENTION

This invention involves nucleic acid-based sensor molecules and methods of generating such nucleic acid-based sensor molecules using in vitro or in vivo selection techniques. The nucleic acid-based sensor molecules of the invention comprise one or more sensor domains and an enzymatic nucleic acid domain. The sensor domain can be linked to the enzymatic nucleic acid domain through one or more oligonucleotides having random sequence (random sequence domain). The sensor domain includes a sequence that is complementary to a specific sequence in a target nucleic acid molecule (effector molecule). Interaction between the sensor domain and the target nucleic acid, i.e., via complementary base-pairing, causes the enzymatic nucleic acid domain to catalyze an enzymatic reaction. The enzymatic reaction can involve a reporter molecule. The reporter molecule can be conjugated to the nucleic acid sensor molecule or can be a separate molecule in the system. In one embodiment, the nucleic acid sensor molecule comprises a sensor domain linked to the enzymatic nucleic acid domain through two oligonucleotides having random sequence. In one embodiment, the sensor domain of the nucleic acid sensor molecule is flanked by an oligonucleotide having a region of random sequence wherein each of the oligonucleotide random sequence domains flanking the sensor domain is linked to a portion of the enzymatic nucleic acid domain.

In one embodiment, the nucleic acid sensor molecule comprises more than one sensor domains. For example, in one embodiment, the nucleic acid sensor molecule can comprise two sensor domains capable of interacting with different regions of the same target nucleic acid sequence. In another embodiment, the nucleic acid sensor molecule can comprise two sensor domains capable of interacting with two target nucleic acid sequences that can be the same or different. Thus, in one embodiment the nucleic acid sensor molecule can comprise two sensor domains capable of interacting with different regions of the same target nucleic acid sequence, an enzymatic nucleic acid domain, and a reporter/signal molecule. In another embodiment, the nucleic acid sensor molecule can comprise two sensor domains capable of interacting with two target nucleic acid sequences that can be the same or different, an enzymatic nucleic acid domain, and a reporter/signal molecule.

The method of the invention relates to evolving nucleic acid sensor molecules whose activity is dependent on interaction between the sensor domain of a nucleic acid sensor and one or more specific sequences in a target nucleic acid molecule (effector molecule). The method of the invention specifically involves creating a candidate mixture of nucleic acid sequences comprising a sensor domain and an enzymatic nucleic acid domain. The sensor domain can be linked to the enzymatic nucleic acid domain through one or more oligonucleotides having random sequence. The sensor domain includes a sequence that is complementary to a specific sequence in a target nucleic acid molecule (effector molecule). The candidate mixture of nucleic acid sequences of the present invention is contacted with a system having the target nucleic acid molecule (effector molecule) under conditions suitable for the interaction of the sensor domain and the complementary sequence in the target nucleic acid molecule. This interaction causes the enzymatic nucleic acid domain to catalyze a reaction that can involve a reporter molecule. Catalytically active nucleic acid sensor molecules are then amplified to yield an enriched mixture of active nucleic acid sequences. These steps are repeated as necessary to identify nucleic acid sensor molecule(s) with specific activity in the presence of the target nucleic acid molecule. In one embodiment, the candidate mixture of nucleic acid sequences can comprise a sensor domain flanked by an oligonucleotide having a region of random sequence (random sequence domain), wherein each of the oligonucleotide random sequence domains flanking the sensor domain is linked to a portion of the enzymatic nucleic acid domain.

The present invention also relates to nucleic acid-based molecular sensors whose activity can be modulated by the presence or absence of nucleic acids, polynucleotides and/or oligonucleotides in a system.

The invention also relates to methods of generating nucleic acid sensor molecules capable of detecting specific target nucleic acids, polynucleotides and/or oligonucleotides in a system. The invention further relates to a method for the detection of specific target signaling molecules, such as nucleic acid molecules, polynucleotides, and/or oligonucleotides, in a variety of analytical settings, including clinical, industrial, veterinary, genomics, environmental, and agricultural applications. The invention further relates to the use of the nucleic acid sensor molecule as molecular sensors capable of modulating the activity, function, or physical properties of other molecules in the presence or absence of target nucleic acids, polynucleotides, and/or oligonucleotides.

The invention further relates to the use of nucleic acid sensor molecules in a diagnostic application to identify the presence of a target nucleic acid molecule, such as a gene and/or gene products, which are indicative of a particular genotype and/or phenotype, for example, a disease state, infection, or related condition within patients or patient samples. The invention also relates to a method for the diagnosis of disease states or physiological abnormalities related to the expression of viral, bacterial or cellular RNA and/or DNA.

Diagnostic applications of the nucleic acid sensor molecules include the use of the nucleic acid sensor molecules for diagnosis of disease, prognosis of therapeutic effect and/or dosing of a drug or class of drugs, prognosis and monitoring of disease outcome, monitoring of patient progress as a function of an approved drug or a drug under development, patient surveillance and screening for drug and/or drug treatment. Diagnostic Such diagnostic applications include the use of nucleic acid sensors for research, development and commercialization of products for the rapid detection of nucleic acids, polynucleotides, and/or oligonucleotides in humans and animals.

Nucleic acid sensor molecules can also be used in assays to assess the specificity, toxicity and effectiveness of various small molecules, nucleoside analogs, or non-nucleic acid drugs against validated targets or biochemical pathways. Nucleic acid sensor molecules can also be used in assays to assess doses of a specific small molecule, nucleoside analog or nucleic acid and non-nucleic acid drug against validated targets or biochemical pathways. The nucleic acid sensors can be used in assays involving in high-throughput screening, biochemical assays, including cellular assays, in vivo animal model assays, clinical trial management, and for mechanistic studies in human clinical studies. The nucleic acid sensor can also be used for the detection of pathogens and biologics, including bio-weapons, in humans, plants, animals or samples therefrom, in connection with environmental testing, detection of biohazards, and/or population screening. The instant invention also features the use of the nucleic acid sensor molecules in other applications, such as functional genomics, target validation and discovery, agriculture or diagnostics, for example the diagnosis of disease or the prevention and/or treatment of human or animal disease.

In one embodiment, the separate binding domains of a nucleic acid sensor molecule of the invention can be distinct separate sequences separated by a linker molecule, or can be a single nucleic acid sequence with distinct hybridization domains.

The use of nucleic acid sensor molecules with more than one sensor domain can be used to detect the presence of two separate target sequences within the same nucleic acid target molecule, such as for greater specificity, or can be used to detect two or more separate nucleic acid molecules independently or as a bifunctional screening agent where the presence of more than one target nucleic acid molecule is required for activity or inactivity of the nucleic acid sensor molecule.

In one embodiment, the K_{d} between any sensor domain of a nucleic sensor molecule of the invention and a nucleic acid target molecule or portion of a nucleic acid target molecule of the invention is between about 1×10⁻¹ and about 1×10⁻³⁰. In another embodiment, the K_{d} between any sensor domain of a nucleic sensor molecule of the invention and a nucleic acid target molecule or portion of a nucleic acid target molecule of the invention is between about 1×10⁻⁵ and about 1×10⁻²⁰. Detection of reporter molecules of the invention can be accomplished using technologies well known in the art. For example, in vivo diagnostic imaging using positron emission tonometry (PET) or magnetic resonance imaging (MRI) techniques can be readily combined with the use of nucleic acid sensor molecules of the invention to detect the presence of cellular, viral, or microbial gene products associated with a disease, infection, or other condition within a patient.

In another embodiment, significant changes in the reporter function of nucleic acid sensor molecules of the invention comprise chemical or physical changes of reporter molecules described herein. For example, a significant change in reporter function can comprise a change in background activity of the nucleic acid sensor molecule greater than 50% over the background activity of the molecule. Alternately, a significant change in reporter function can comprise a change in background activity of the nucleic acid sensor molecule of between 10 and 1×10¹⁰ fold, such as 1×10⁴, 1×10⁵, 1×10⁶, 1×10⁷, or 1×10⁸ fold.

Additional diagnostic applications of nucleic acid sensor moleculeswithin the scope of the invention include their use as in vivo imaging agents. For example, such imaging agents are useful in detecting various cancers by detecting neoplastic gene products expressed by tumors. Alternately, imaging agents comprising nucleic acid sensor molecules of the invention can be.

The reagents and methods of the invention can be used to detect cellular, viral, or microbial gene products produced by infection or by other pathological conditions in a patient. The nucleic acid sensor molecules can be readily adapted for use in various in vivo imaging technologies known in the art. For example, the use of nucleic acid sensor molecules and/or reporter molecules containing various labels, such as radioisotopes, can be used in conjunction with positron emission tonometry (PET), or magnetic resonance imaging (MRI) techniques to detect cellular, viral, or microbial gene products associated with a particular disease, infection or condition in a patient. the presence or absence of target nucleic acid molecules in a simple or complex sample. The method also can be used in an assay to detect a multitude of different nucleic acid molecules in a sample, for example a blood sample from a patient. The method also can be used in array and multi-array detection formats, for example chip-based and microtiter-based detection platforms comprising a plurality of specific nucleic acid sensor molecules. These detection formats are used for molecular profiling and genome scoring, useful in a variety of applications, such as for customized therapeutic approaches to patient care and treatment.

### BRIEF DESCRIPTION OF THE DRAWINGS

### DRAWINGS

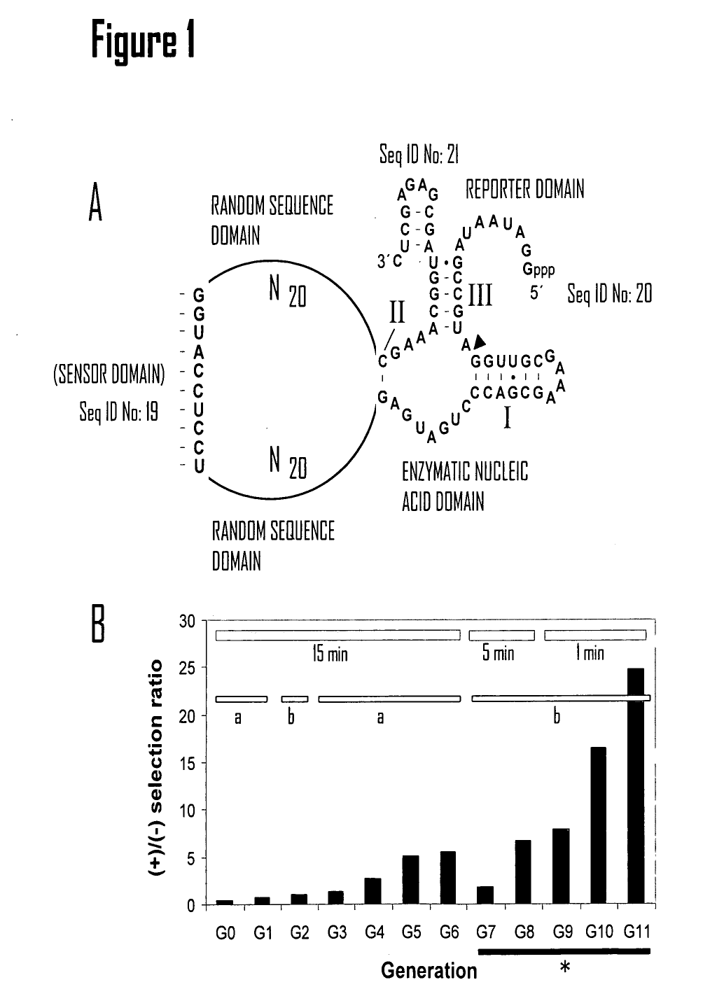

FIGS. 1A and B show a non-limiting example of the in vitro selection of oligonucleotide-sensitive enzymatic nucleic acid molecules. (A) Shows an example of a construct used to initiate the in vitro selection process. (B) Shows a plot depicting the ratio of enzymatic nucleic acid activity in the presence (+) vs. the absence (−) of an 18-nucleotide effector molecule.

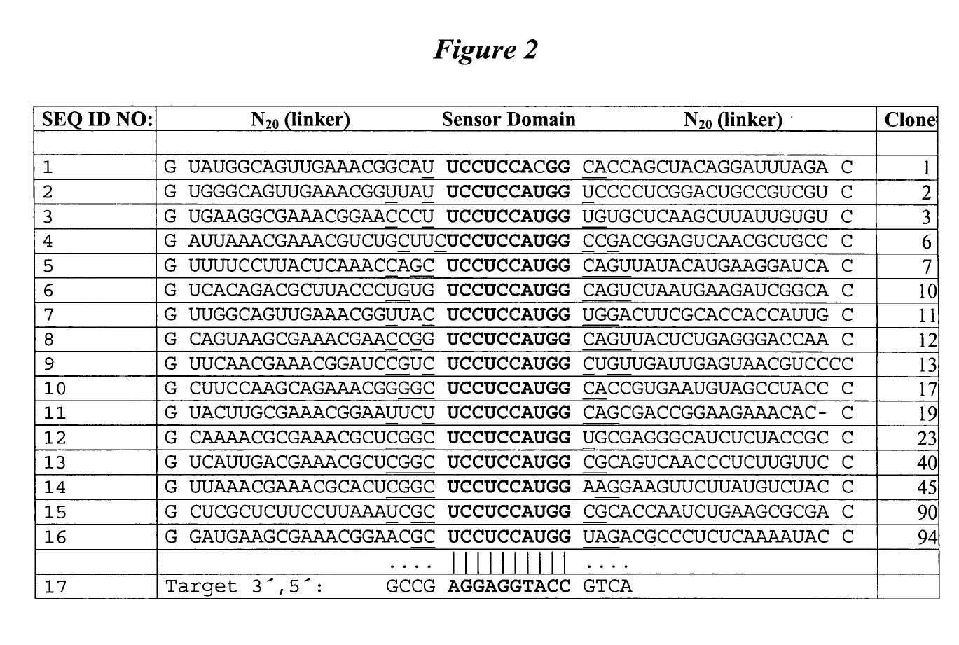

FIG. 2 shows non-limiting examples of the allosteric domain sequences of nucleic acid based-nucleic acid sensor molecules generated by the method of the instant invention.

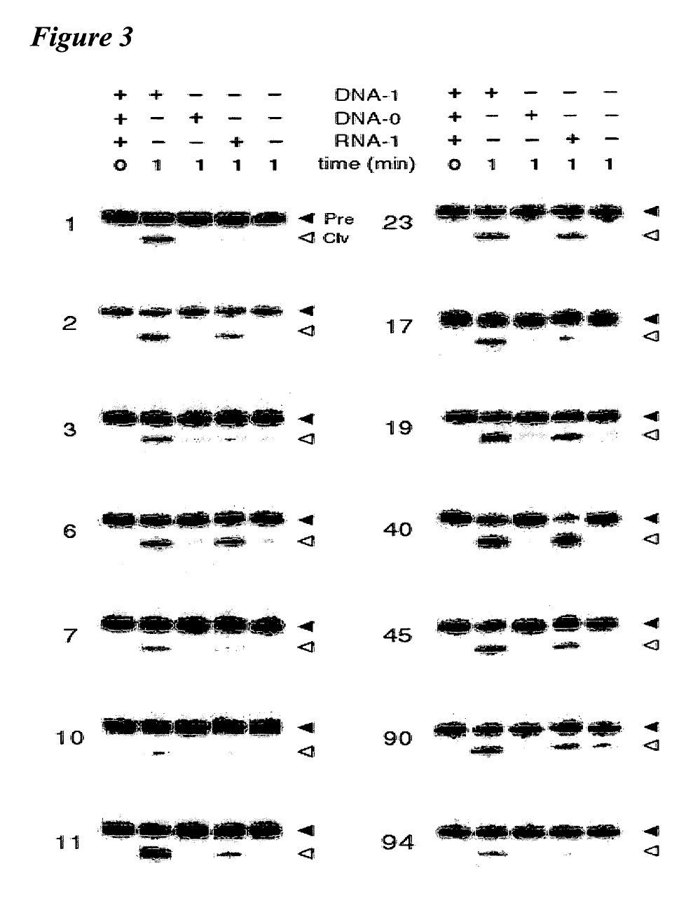

FIG. 3 shows the results of a non-limiting example of an activity assay for fourteen oligonucleotide-sensitive nucleic acid sensor molecules of the invention. Each nucleic acid sensor molecule was incubated under in vitro selection conditions for the time indicated in the absence of target (lane 5) or in the presence of various target oligonucleotides as indicated. DNA-1 is the matched DNA molecule, DNA-0 is a non-complementary DNA oligomer, and RNA-1 is the RNA version of DNA-1. “Pre” refers to precursor and “Clv” refers to cleavage product.

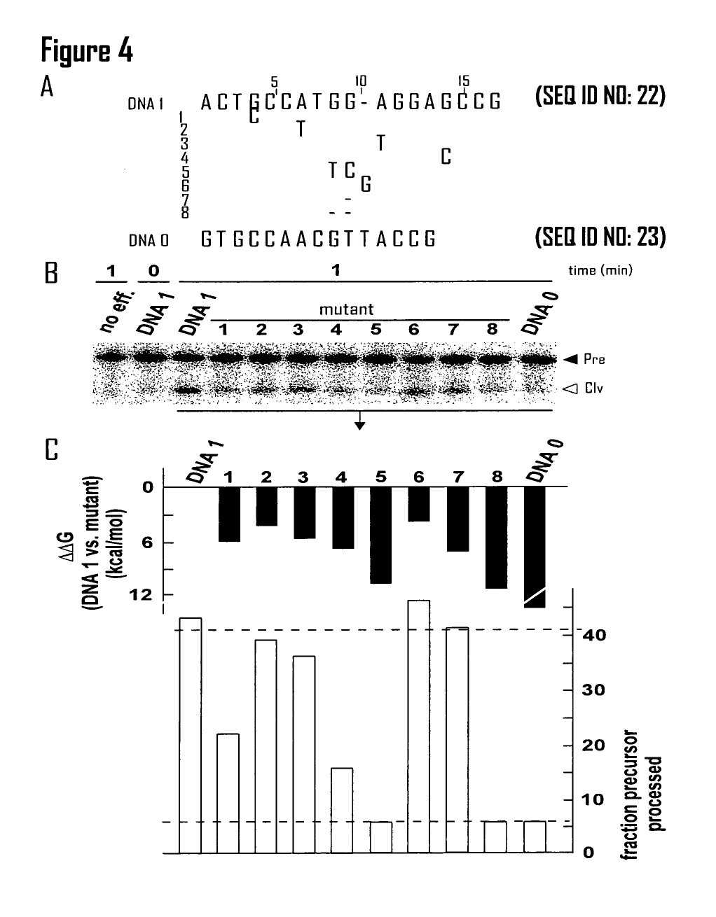

FIGS. 4A-C show the results of a non-limiting example of effector specificity of a nucleic acid based sensor molecule of the invention. The level and type of discrimination exhibited by the nucleic acid sensor molecule was examined by testing the activity of the nucleic acid sensor molecule against a series of closely related mutants of the DNA 1 sequence. FIG. 4A shows the sequence of the original effector DNA (DNA 1), the sequences of eight closely related mutants of the DNA 1 sequence (1-8), and the sequence of the mismatched DNA oligonucleotide (DNA 0). FIG. 4B shows the results of the activity assay using DNA 1, DNA 0 and the various DNA 1 mutant sequences as substrate. Activity was measured by gel analysis of a 5′ 32P-labeled sensor after incubation under in vitro selection conditions for the times indicated in the absence of effector or in the presence of various target oligonucleotides as indicated. FIG. 4C is a plot showing the differences in the free energy of duplex formation between the DNA 1 complex with sensor versus the free energy of duplex formation between the various mutant DNAs and the sensor (Top) and the fraction of sensor precursor cleaved in the presence of each effector DNA as indicated (Bottom).

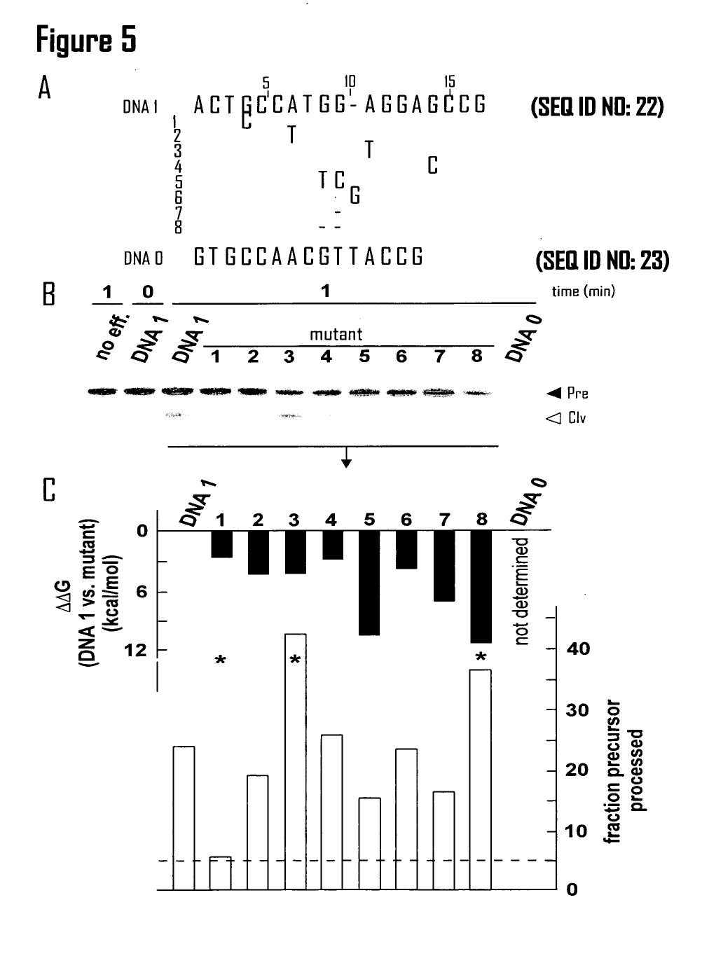

FIGS. 5A-C show the results of a non-limiting example of effector specificity of another nucleic acid sensor molecule of the invention. FIG. 5A shows the sequence of the original effector DNA (DNA 1), the sequences of eight closely related mutants of the DNA 1 sequence (1-8), and the sequence of the mismatched DNA oligonucleotide (DNA 0). FIG. 5B shows the results of the activity assay using DNA 1, DNA 0 and the various DNA 1 mutant sequences as substrate. Activity was measured as described in FIG. 4B. FIG. 5C is a plot showing the differences in the free energy of duplex formation between the DNA 1 complex with sensor versus the free energy of duplex formation between the various mutant DNAs and the sensor (Top) and the fraction of sensor precursor cleaved in the presence of each effector DNA as indicated (Bottom).

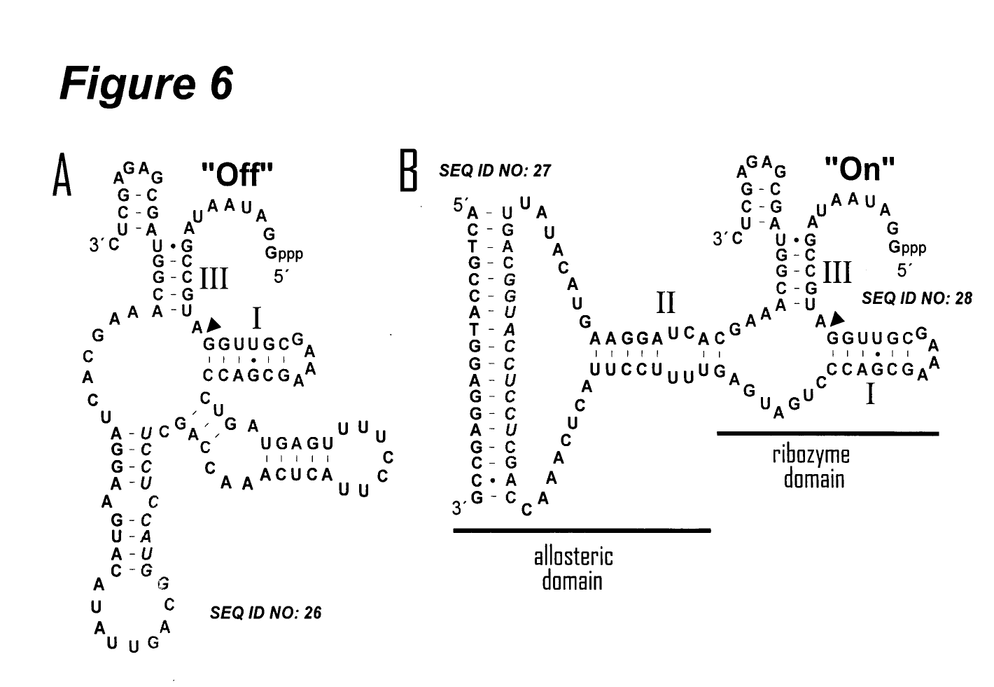

FIGS. 6A and B show non-limiting structural models indicating potential structures of inactive and active conformations of a nucleic acid sensor molecule of the invention.

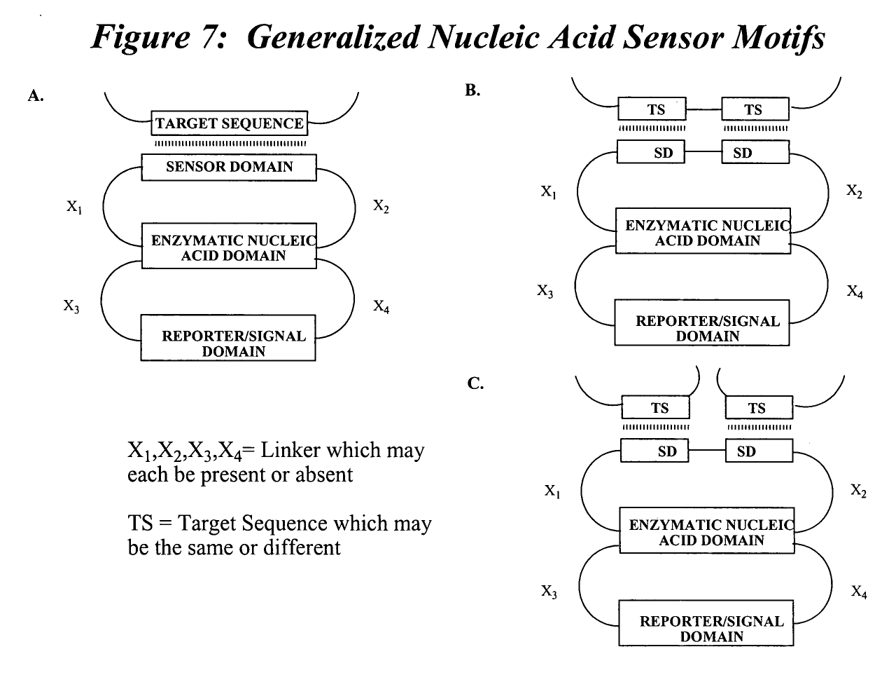

FIGS. 7A-C show non-limiting examples of generalized nucleic acid sensor molecule constructs of the invention. (A) shows a nucleic acid sensor construct comprising a sensor domain, an enzymatic nucleic acid domain, and a reporter/signal molecule. (B) shows a nucleic acid sensor construct comprising two sensor domains capable of interacting with different regions of the same target nucleic acid sequence, an enzymatic nucleic acid domain, and a reporter/signal molecule. (C) shows a nucleic acid sensor construct comprising two sensor domains capable of interacting with two target nucleic acid sequences that can be the same or different, an enzymatic nucleic acid domain, and a reporter/signal molecule. Regions X1, X2, X3, and X4 all represent linker molecules (nucleotide and/or non-nucleotide) that can each be present or absent.

### DETAILED DESCRIPTION OF THE INVENTION

In one embodiment, the invention features a method comprising: (a) generating a candidate mixture of nucleic acid sequences comprising a sensor domain, a random sequence domain, and an enzymatic nucleic acid domain, wherein the sensor domain has a sequence complementary to a predetermined sequence in a target nucleic acid molecule and is flanked on either side by a random sequence domain and wherein each of the random sequence domains is linked to a portion of an enzymatic nucleic acid domain; (b) contacting the candidate mixture of nucleic acid sequences from (a) with the target nucleic acid molecule under conditions suitable for the target nucleic acid molecule to interact with the sensor domain of an active nucleic acid sequence of the candidate mixture and activate the enzymatic nucleic acid domain of such active nucleic acid sequence of the candidate mixture to catalyze a reaction involving a reporter molecule; (c) partitioning the active nucleic acid sequences from the rest of the candidate mixture; (d) amplifying the active nucleic acid sequences to yield an enriched mixture of active nucleic acid sequences; and (e) repeating steps (b)-(d), as necessary, to identify a nucleic acid sensor molecule capable of catalyzing a chemical reaction in the presence of the target nucleic acid molecule. The reporter molecule can be attached to the nucleic acid sequences in the candidate mixture or can be present in the candidate mixture as a separate molecule.

In one embodiment, the invention features a method comprising: (a) generating a candidate mixture of nucleic acid sequences comprising a sensor domain, a random sequence domain, and an enzymatic nucleic acid domain, wherein the sensor domain has a sequence complementary to a predetermined sequence in a target nucleic acid molecule and wherein the sensor domain is flanked on either side by a random sequence domain and each of the random sequence domains is linked to a portion of an enzymatic nucleic acid domain; (b) contacting the candidate mixture of nucleic acid sequences from (a) with the target nucleic acid molecule under conditions suitable for the target nucleic acid molecule to interact with the sensor domain of an active nucleic acid sequence of the candidate mixture and inactivate the ability of the enzymatic nucleic acid domain of such active nucleic acid sequence of the candidate mixture to catalyze a reaction involving a reporter molecule; (c) partitioning the inactive nucleic acid sequences from the rest of the candidate mixture; (d) amplifying the inactive nucleic acid sequences to yield an enriched mixture of inactive nucleic acid sequences; and (e) repeating steps (b)-(d), as necessary, to identify a nucleic acid sensor molecule capable of catalyzing a chemical reaction only in the absence of the target nucleic acid molecule. The reporter molecule can be attached to the nucleic acid sequences in the candidate mixture or can be present in the candidate mixture as a separate molecule

In another embodiment, the invention features a method comprising: (a) generating a candidate mixture of nucleic acid sequences comprising a sensor domain a random sequence domain, and an enzymatic nucleic acid domain, wherein the sensor domain has a sequence complementary to a predetermined sequence in a target nucleic acid molecule and wherein the sensor domain is flanked on either side by a random sequence domain and each of the random sequence domains is linked to a portion of an enzymatic nucleic acid domain; (b) contacting the candidate mixture of nucleic acid sequences from (a) with the target nucleic acid molecule under conditions suitable for the target nucleic acid molecule to interact with the sensor domain of an active nucleic acid sequence of the candidate mixture and activate the enzymatic nucleic acid domain of such active nucleic acid sequence of the candidate mixture to catalyze a reaction involving a reporter molecule; (c) partitioning the active nucleic acid sequences from the rest of the candidate mixture; (d) amplifying the active nucleic acid sequences to yield an enriched mixture of active nucleic acid sequences; (e) repeating steps (b)-(d), as necessary, to identify a nucleic acid sensor molecule capable of catalyzing a chemical reaction in the presence of the target nucleic acid molecule; and (f) modifying the nucleic acid sensor molecule of (e), wherein the modification comprises adding, deleting, or substituting nucleotide residues and wherein such modification maintains the requirement of target nucleic acid molecule interaction for the catalytic activity of the nucleic acid sensor molecule. The reporter molecule can be attached to the nucleic acid sequences in the candidate mixture or can be present in the candidate mixture as a separate molecule.

In another embodiment, the invention features a method comprising: (a) generating a candidate mixture of nucleic acid sequences comprising a sensor domain a random sequence domain, and an enzymatic nucleic acid domain, wherein the sensor domain has a sequence complementary to a predetermined sequence in a target nucleic acid molecule and wherein the sensor domain is flanked on either side by a random sequence domain and each of the random sequence domains is linked to a portion of an enzymatic nucleic acid domain; (b) contacting the candidate mixture of nucleic acid sequences from (a) with the target nucleic acid molecule under conditions suitable for the target nucleic acid molecule to interact with the sensor domain of an active nucleic acid sequence of the candidate mixture and inactivate the enzymatic nucleic acid domain of such active nucleic acid sequence of the candidate mixture; (c) partitioning the inactive nucleic acid sequences from the rest of the candidate mixture; (d) amplifying the inactive nucleic acid sequences to yield an enriched mixture of inactive nucleic acid sequences; (e) repeating steps (b)-(d), as necessary, to identify a nucleic acid sensor molecule capable of catalyzing a chemical reaction involving a reporter molecule in the absence of the target nucleic acid molecule; and (f) modifying the nucleic acid sensor molecule of (e), wherein the modification comprises adding, deleting, or substituting nucleotide residues and wherein such modification maintains the catalytic activity of the nucleic acid sensor molecule in the absence of the target nucleic acid molecule. The reporter molecule can be attached to the nucleic acid sequences in the candidate mixture or can be present in the candidate mixture as a separate molecule.

In one embodiment, the selected nucleic acid sensor molecule is modified. In one embodiment, the modification comprises deleting one or more nucleotides from the nucleic acid sensor molecule. In another embodiment, the modification comprises adding one or more nucleotides to the nucleic acid sensor molecule. In another embodiment, the modification comprises substituting one or more nucleotides of the nucleic acid sensor molecule with another nucleotide. In all of the above modifications, the overall property (activity or inactivity) of the nucleic acid sensor molecule in the presence of a target nucleic acid molecule is retained following such modification. Such modification can be used, for example, to shorten or lengthen the nucleic acid sensor molecule while retaining the desired property of the sensor molecule. In addition, such modification can be used, for example, to stabilize the nucleic acid sensor molecule from nuclease degradation or improve the overall catalytic activity of the sensor molecule. The nucleotide residues which are added or substituted when modifying a nucleic acid sensor molecule in a method of the invention can comprise chemically modified nucleotides, for example nucleic acid backbone, nucleic acid sugar, and/or nucleic acid base-modified nucleotides.

In one embodiment of the inventive method, the nucleic acid molecules making up the candidate mixture of the invention comprise more than one sensor domain which has sequences complementary to the same or different sequences within the same or different target nucleic acid molecule(s). When more than one sensor domain is present, the sensor domains can be for the same target nucleic acid sequence or for different target nucleic acid binding sequences, for example sequences of equal or unequal nucleotide composition and/or length.

In one embodiment, the sensor domain(s) in the candidate mixture of nucleic acid sequence used in the invention is from about 4 to about 100 nucleotides in length. In another embodiment, the sensor domain(s) is from about 8 to about 50 nucleotides in length. In yet another embodiment, the sensor domain(s) is from about 10 to about 20 nucleotides in length.

In one embodiment, the nucleic acid molecules making up the candidate mixture of the invention comprise two random sequence domains that link the sensor domain to the enzymatic nucleic acid domain. The random sequence domains can be of equal or unequal length. In one embodiment, the random sequence region in the random sequence domain is from about 4 to about 200 nucleotides in length. In another embodiment, the random sequence domain is from about 10 to about 50 nucleotides in length.

In one embodiment of the inventive method, the partitioning of active nucleic acid sensor sequences comprises selection of active nucleic acid sequences based on a reaction catalyzed by the enzymatic nucleic acid sensor molecule. Activity of the enzymatic nucleic acid sensor molecule can comprise a cleavage or ligation reaction, for example, cleavage or ligation of a nucleic acid reporter molecule. The reporter molecule can be attached to the nucleic acid sequences in the candidate mixture or can be a separate molecule in the system. Examples of reporter molecules include nucleic acid molecules comprising various tags, probes, beacons, fluorophores, chemophores, ionophores, radio-isotopes, photophores, peptides, proteins, enzymes, antibodies, nucleic acids, and enzymatic nucleic acids or a combination thereof. Determination of cleavage or ligation can be accomplished by any method, such as those known in the art, including chemical or physical signals produced or inhibited by activity of the nucleic acid sensor molecule. Non-limiting examples of such chemical and physical signals include changes in fluorescence such as FRET (fluorescent resonance energy transfer), color change, UV absorbance, phosphorescence, pH, optical rotation, isomerization, polymerization, temperature, mass, capacitance, resistance, and emission of radiation.

Nucleic acid sensor molecules are isolated from the pool of sequences (candidate mixture) that have activity in the presence of or absence of the target nucleic acid sequence. These nucleic acid sensor sequences are subsequently amplified. Amplification can be accomplished through a variety of methods, including via PCR amplification and other amplification methods known in the art. The process can be repeated until sensor molecules are identified with a desired level of activity. Furthermore, mutagenesis of the amplified sequences, for example via mutagenic PCR (see for example Kore et al., 2000, J. Mol. Biol., 301, 1113-1121), can be used to provide more diversity than the original pool of nucleic acid molecules has provided.

Furthermore, in any of the above methods, the steps can be carried out more than once. Thus, in one embodiment, the method of the instant invention is carried out more than once.

The methods of the invention are used to provide nucleic acid sensor molecules capable of detecting the presence or absence of one or more target nucleic acid molecules in a system. In one embodiment, detection of the target nucleic acid sequence is indicative of the presence of the predetermined target nucleic acid sequence in the system. In another embodiment, lack of detection of the target nucleic acid sequence in the system is indicative of the absence of the target nucleic acid sequence in the system.

In one embodiment, a system of the invention is an in vitro system, for example, a sample derived from the group consisting of an organism, mammal, patient, plant, water, beverage, food preparation, and soil.

In another embodiment, a system of the invention is an in vivo system, for example a bacteria, a bacterial cell, a fungus, a fungal cell, a virus, a plant, plant cell, mammal, mammalian cell, human, or human cell.

In another embodiment, the method of the invention is used to isolate nucleic acid sensor molecules that detect the presence of or absence of target nucleic acids, such as polynucleotides, and/or oligonucleotides, in a test sample, for example, in a blood sample, serum sample, urine sample, or other tissue sample, cell extract, cell, tissue extract, or entire organism.

The present invention can be used, for example, to indicate the presence of a viral nucleic acid in a test sample. Thus, in one embodiment of the inventive method the target nucleic acid (or polynucleotide or oligonucleotide) is a viral DNA or RNA, such as that derived from HCV, HBV, HIV, HPV, HTLV-1, CMV, HSV, RSV, Rhinovirus, WNV, Hantavirus, Ebola virus, smallpox virus or Encephalovirus.

The present invention can also be used, for example, to indicate the presence of a bacterial nucleic acid in a test sample. Thus, in one embodiment of the inventive method the target nucleic acid (or polynucleotide or oligonucleotide) is a bacterial DNA or RNA, such as that derived from anthrax (bacillus anthracus), E-coli, streptococcus, listeria, salmonella, campylobacter, enterobacter, psuedomonas, staphylococcus, or coliform.

The present methods also contemplate arrays of nucleic acid sensor molecules, for example when attached to a surface such as a chip or bead, that can be used to detect and profile target nucleic acids, polynucleotides, and/or oligonucleotides in a system. For example, such arrays can be used for differential gene expression analysis and/or SNP detection, for verification of known sequences, for fingerprinting nucleic acid polymers, and for mapping homologous segments within a nucleic acid sequence (see for example U.S. Pat. No. 6,303,301 and U.S. Pat. No. 6,306,643).

In another embodiment, nucleic acid sensor molecules of the invention are derived from random pools of nucleic acid molecules under selective pressure (see for example U.S. Pat. No. 5,817,785, U.S. Pat. No. 5,475,096, and U.S. Pat. No. 5,270,163 incorporated by reference herein). For example, a nucleic acid sensor molecule can be chosen from a pool of nucleic acid molecules wherein the criteria for selection is the cleavage or ligation of a reporter molecule in the presence of a predetermined nucleic acid, polynucleotide, and/or oligonucleotide. The cleavage or ligation event is assayed for by methods known in the art and as described herein, for example, via fluorescence. Other criteria can be established to accommodate other methods of detection.

The present invention relates to nucleic acid sensor molecules capable of detecting the presence or absence of one or more target nucleic acid molecules in a system. The nucleic acid-based sensor molecule comprises one or more sensor domains and an enzymatic nucleic acid domain. The sensor domain can be linked to the enzymatic nucleic acid domain through one or more oligonucleotides having random sequence (random sequence domain). The sensor domain includes a sequence that is complementary to a specific sequence in a target nucleic acid molecule (effector molecule). Interaction between the sensor domain and the target nucleic acid, i.e., via complemetary base-pairing, causes the enzymatic nucleic acid domain to catalyze an enzymatic reaction. The enzymatic reaction can involve a reporter molecule. In one embodiment, the nucleic acid sensor molecule is capable of catalyzing a chemical reaction in the presence of the target nucleic acid molecule. In another embodiment, the nucleic acid sensor molecule is capable of catalyzing a chemical reaction in the absence of the target nucleic acid molecule.

In one embodiment, the nucleic acid sensor molecule comprises more than one sensor domain. In one embodiment, the nucleic acid sensor molecule can comprise more than one sensor domain which has sequences complementary to the same or different sequences within the same or different target nucleic acid molecule(s). When more than one sensor domain is present, the sensor domains can be for the same target nucleic acid sequence or for different target nucleic acid binding sequences, for example sequences of equal or unequal nucleotide composition and/or length. In one embodiment, the sensor domain(s) of the nucleic acid sensor molecule is from about 4 to about 100 nucleotides in length. In another embodiment, the sensor domain(s) is from about 8 to about 50 nucleotides in length. In yet another embodiment, the sensor domain(s) is from about 10 to about 20 nucleotides in length.

In one embodiment, the nucleic acid sensor molecule comprises more than one oligonucleotide having random sequence. In one embodiment, the nucleic acid sensor molecule comprises two random sequence domains that link the sensor domain to the enzymatic nucleic acid domain. The random sequence domains can be of equal or unequal length. In one embodiment, the random sequence region in the random sequence domain is from about 4 to about 200 nucleotides in length. In another embodiment, the random sequence domain is from about 10 to about 50 nucleotides in length.

The invention features nucleic acid sensor molecules as described throughout the specification and as described above in the inventive methods. In one embodiment, the invention features a nucleic acid sensor molecule having a sensor domain comprising SEQ ID NOS: 1-16. In another embodiment, the invention features a nucleic acid sensor molecule having a sensor domain comprising SEQ ID NOS: 1-16.

In another embodiment, the enzymatic nucleic acid domain of the nucleic acid sensor molecule is derived from a hammerhead, inozyme, g-cleaver, hairpin, Zinzyme, Amberzyme, or DNAzyme motif.

Nucleic acid sensor molecules of the invention that are used to detect the presence of the target nucleic acid molecules(s) can have a detection signal, such as from a reporter molecule. Examples of such reporter molecules include nucleic acid molecules comprising various tags, probes, beacons, fluorophores, chemophores, ionophores, radio-isotopes, photophores, peptides, proteins, enzymes, antibodies, nucleic acids, and enzymatic nucleic acids or a combination thereof. The reporter molecule may optionally be covalently linked to a portion of the nucleic acid sensor molecule.

In one embodiment, the present invention features a nucleic acid sensor molecule comprising an enzymatic nucleic acid component and one or more sensor components where, in

response to an interaction of a target nucleic acid molecule with the sensor component, the enzymatic nucleic acid component catalyzes a chemical reaction. The chemical reaction is generally on a reporter molecule which can be conjugated to the nucleic acid sensor molecule or can be a separate molecule in the system. Preferably, the chemical reaction in which the activity or physical properties of a reporter molecule is modulated results in a detectable response. Thus, in one embodiment of the inventive method, the presence of a detectable response, for example, the detection of a chemical reaction indicates the presence of the target, i.e. the target nucleic acid, polynulceotide, or oligonucleotide in the system. In another embodiment, the absence of a detectable response, for example, the absence of a chemical reaction, indicates the lack of the target nucleic acid, polynucleotide, or oligonucleotide in a system.

Examples of reporter molecules contemplated by the instant invention include molecular beacons, small molecules, fluorophores, chemophores, ionophores, radio-isotopes, photophores, peptides, proteins, enzymes, antibodies, nucleic acids, and enzymatic nucleic acids or a combination thereof (see for example in Singh et al., 2000, Biotech., 29, 344; Lizardi et al., U.S. Pat. Nos. 5,652,107 and 5,118,801).

In one embodiment, the nucleic acid sensor molecule can be conjugated with a signal or reporter molecule via a linker. Either the nucleic acid sensor molecule or the signal/reporter molecule, or both can have a linker region. The linker region, when present in the nucleic acid sensor molecule and/or reporter molecule can be comprised of nucleotide, non-nucleotide chemical moieties or combinations thereof. Non-limiting examples of non-nucleotide chemical moieties can include ester, anhydride, amide, nitrile, and/or phosphate groups. In another embodiment, the non-nucleotide linker is as defined herein below and can include, for example, abasic nucleotide, polyether, polyamine, polyamide, peptide, carbohydrate, lipid, or polyhydrocarbon compounds.

Thus, in one embodiment, the invention features a nucleic acid sensor molecule of the invention having one or more non-nucleotide moieties, and having enzymatic activity to perform a chemical reaction, for example to cleave an RNA or DNA molecule.

The reporter molecule of the present invention also can be an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

Using such reporter molecules and others known in the art, the detectable signal or response involving the nucleic acid sensor molecule of the instant invention can be monitored by, for example, a change in fluorescence such as FRET (fluorescent resonance energy transfer), color change, UV absorbance, phosphorescence, pH, optical rotation, isomerization, polymerization, temperature, mass, capacitance, resistance, and emission of radiation.

The nucleic acid sensor molecules can be used to detect the presence of or absence of target nucleic acids, such as polynucleotides, and/or oligonucleotides, in a test sample, for example, in a blood sample, serum sample, urine sample, or other tissue sample, cell extract, cell, tissue extract, or entire organism.

The nucleic acid sensor molecules can also be used, for example, to indicate the presence of a viral nucleic acid in a test sample. Thus, in one embodiment of the inventive method the target nucleic acid (or polynucleotide or oligonucleotide) is a viral DNA or RNA, such as that derived from HCV, HBV, HIV, HPV, HTLV-1, CMV, HSV, RSV, Rhinovirus, WNV, Hantavirus, Ebola virus, smallpox virus or Encephalovirus.

The present invention can also be used, for example, to indicate the presence of a bacterial nucleic acid in a test sample. Thus, in one embodiment of the inventive method the target nucleic acid (or polynucleotide or oligonucleotide) is a bacterial DNA or RNA, such as that derived from anthrax (bacillus anthracus), E-coli, streptococcus, listeria, salmonella, campylobacter, enterobacter, psuedomonas, staphylococcus, or coliform.

In one embodiment, the invention also features an array of nucleic acid sensor molecules comprising a predetermined number of nucleic acid sensor molecules of the invention (see for example U.S. Pat. No. 6,242,246 incorporated by reference herein). In one embodiment, a nucleic acid sensor molecule of the instant invention is attached to a solid surface. For example, the surface can comprise silicon-based chips, silicon-based beads, controlled pore glass, polystyrene, cross-linked polystyrene, nitrocellulose, biotin, plastics, metals and polyethylene films.

Methods and nucleic acid sensor molecules of the invention can be used in genome discovery, detection, and scoring. In a non-limiting example, the method and nucleic acid sensor molecules of the invention can be used to screen a fetus, infant, child or adult's genome. A sample of material is obtained from, for example, amniotic fluid, chorionic villus, blood, or hair and is contacted with an array of nucleic acid sensor molecules. The information generated by the array can be used in diagnostic molecular profiling applications such as genome mapping or profiling for various purposes, for example, in target discovery, target validation, for example, validation of a predetermined RNA target, drug discovery, determining susceptibility to disease, determining the potential effect of various treatments or therapies, predicting drug metabolism or drug response, selecting candidates for clinical trials, and for managing the treatment of disease in individual patients.

In additional embodiments, the invention features a method and nucleic acid sensor molecules for the detection of target nucleic acids, polynucleotides, and/or oligonucleotides in both in vitro and in vivo applications. In vitro diagnostic applications can comprise both solid support based and solution based chip, multichip-array, micro-well plate, and micro-bead derived applications as are commonly used in the art. In vivo diagnostic applications can include but are not limited to cell culture and animal model based applications, comprising differential gene expression arrays, FACS based assays, diagnostic imaging, and others.

Methods and nucleic acid sensor molecules of the invention can be used in a diagnostic application to identify the presence of a target nucleic acid, polynucleotide, or oligonucleotide which is indicative of a particular genotype and/or phenotype, for example, a disease state, infection, or related condition within an organism, such as a patient, or organism (i.e., patent) sample, such as blood, serum, urine, other tissue sample, cell extract, cell, tissue extract, or entire organism.

Methods and nucleic acid sensor molecules of the invention can also be used for the diagnosis of disease states or physiological abnormalities related to the expression of viral, bacterial or cellular nucleic acids, polynucleotides, and/or oligonucleotides.

Methods and nucleic acid sensor molecules of the invention can be used for the diagnosis of disease, prognosis of therapeutic effect and/or dosing of a drug or class of drugs, prognosis and monitoring of disease outcome, monitoring of patient progress as a function of an approved drug or a drug under development, patient surveillance and screening for drug and/or drug treatment. Diagnostic applications include the use of methods and nucleic acid sensor molecules of the invention for research, development and commercialization of products for the rapid detection of macromolecules, such as mammalian viral nucleic acids, for the diagnosis of diseases associated with viruses, prions and viroids in humans and animals.

In one embodiment, a system of the invention is an in vitro system, for example, a sample derived from the group consisting of an organism, mammal, patient, plant, water, beverage, food preparation, and soil.

In another embodiment, a system of the invention is an in vivo system, for example a bacteria, a bacterial cell, a fungus, a fungal cell, a virus, a plant, plant cell, mammal, mammalian cell, human, or human cell.

By “nucleic acid sensor molecule” or “allozyme” as used herein is meant a nucleic acid molecule comprising an enzymatic domain and a sensor domain, where the enzymatic nucleic acid domain's ability to catalyze a chemical reaction involving a reporter molecule is dependent on the interaction with a target nucleic acid, polynucleotide, or oligonucleotide. The introduction of chemical modifications, additional functional groups, and/or linkers, to the nucleic acid sensor molecule can provide enhanced catalytic activity of the nucleic acid sensor molecule, increased binding affinity of the sensor domain to a target nucleic acid, and/or improved nuclease/chemical stability of the nucleic acid sensor molecule, and are hence within the scope of the present invention (see for example Usman et al., U.S. patent application Ser. No. 09/877,526 incorporated by reference herein, Usman et al., WO 01/66721, George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842).

By “enzymatic nucleic acid” is meant a nucleic acid molecule capable of catalyzing (altering the velocity and/or rate of) a variety of reactions including the ability to cleave other separate nucleic acid molecules (endonuclease activity) or ligate other separate nucleic acid molecules (ligation activity) in a nucleotide base sequence-specific manner. Additional reactions amenable to nucleic acid sensor molecules include, but are not limited to, phosphorylation, dephosphorylation, isomerization, helicase activity, polymerization, transesterification, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reactions on both small molecules and macromolecules. Such a molecule with endonuclease and/or ligation activity can have nucleotide complementarity in a substrate binding region of the molecule to a specified nucleic acid sequence (reporter/signaling molecule), and also has an enzymatic activity that specifically cleaves and/or ligates RNA or DNA in that reporter nucleic acid sequence. That is, the nucleic acid molecule with endonuclease and/or ligation activity is able to intramolecularly or intermolecularly cleave and/or ligate RNA or DNA and thereby inactivate or activate a reporter RNA or DNA molecule. This complementarity functions to allow sufficient hybridization of the enzymatic nucleic acid molecule to the reporter RNA or DNA to allow the cleavage/ligation to occur. 100% complementarity is preferred, but complementarity as low as 50-75% can also be useful in this invention. In addition, nucleic acid sensor molecule can perform other reactions, including those mentioned above, selectively on both small molecule and macromolecular substrates, though specific interaction of the nucleic acid sensor molecule sequence with the desired substrate molecule or reporter molecule, for example via hydrogen bonding, electrostatic interactions, and Van der Waals interactions. The nucleic acids can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity.

Several varieties of enzymatic nucleic acids are known presently, which can catalyze, for example, the hydrolysis of RNA phosphodiester bonds in trans (and thus can cleave other RNA molecules) under physiological conditions, for example trans-cleaving or trans-splicing ribozymes. In general, enzymatic nucleic acids with RNA endonuclease activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid, for example, first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the enzymatic nucleic acid is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of an enzymatic nucleic acid molecule.

By “random sequence domain” is meant a particular sequence of fixed or varying length, comprising nucleotides or mixed or random sequence.

By “signal” or “reporter molecule” as described herein, is meant a molecule, such as a nucleic acid sequence (e.g., RNA or DNA or analogs thereof) or peptides and/or other chemical moieties, able to stably interact with the nucleic acid sensor molecule and function as a substrate for the enzymatic nucleic acid domain of the nucleic acid sensor molecule. The reporter molecule can contain chemical moieties capable of generating a detectable response, including, but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system. The reporter molecule of the invention can be, for example, an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” or “sensor domain” of the nucleic acid sensor molecule as used herein is meant, a nucleic acid sequence (e.g., RNA or DNA or analogs thereof) which interacts with a nucleic acid sequence in one or more regions of a target nucleic acid molecule or more than one target nucleic acid molecules, and which interaction causes the enzymatic nucleic acid component of the nucleic acid sensor molecule to either catalyze a reaction or stop catalyzing a reaction involving a reporter molecule. In the presence of a nucleic acid used for detection and/or a target nucleic acid, polynucleotide, and/or oligonucleotide of the invention, the ability of the sensor component, for example, to modulate the catalytic activity of the nucleic acid sensor molecule, is inhibited or diminished. The sensor component can comprise recognition properties relating to chemical or physical signals capable of modulating the nucleic acid sensor molecule via chemical or physical changes to the structure of the nucleic acid sensor molecule. The sensor component can be derived from a naturally occurring nucleic acid binding sequence, for example, RNAs that bind to other nucleic acid sequences in vivo. Alternately, the sensor component can be derived from a nucleic acid molecule (aptamer) which is evolved to bind to a nucleic acid sequence within a target nucleic acid molecule. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

By “sufficient length” is meant an oligonucleotide of length sufficient to provide the intended function (such as binding) under the expected condition. For example, a binding arm of the enzymatic nucleic acid component of the nucleic acid sensor molecule should be of “sufficient length” to provide stable binding to the reporter molecule under the expected reaction conditions and environment to catalyze a reaction. In a further example, the sensor domain of the nucleic acid sensor molecule should be of sufficient length to interact with a target nucleic acid molecule in a manner that would cause the nucleic acid sensor to be active.

By “nucleic acid molecule” as used herein is meant a molecule comprising nucleotides. The nucleic acid can be single, double, or multiple stranded and can comprise modified or unmodified nucleotides or non-nucleotides such as protein nucleic acids (PNA) or locked nucleic acids (LNA) or various mixtures and combinations thereof. Nucleic acid molecules include oligonucleotides, ribozymes, DNAzymes, templates, primers, nucleic acid sensor molecules, reporter and or signal molecules.

By “target nucleic acid molecule” is meant a molecule comprising nucleotides that are capable of interacting with the sensor domain of a nucleic acid sensor molecule of the invention in a manner that causes the nucleic acid sensor molecule to have catalytic activity.

By “oligonucleotide” is meant a nucleic acid molecule comprising a stretch of three or more nucleotides.

The term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine. The terms “abasic” or “abasic nucleotide” are meant to include sugar moieties lacking a base or having other chemical groups in place of a base at the 1′ position, (for more details see Wincott et al., International PCT publication No. WO 97/26270). The term non-nucleotide, i.e., such as a non-nucleotide linker of a nucleic acid sensor molecule and/or reporter molecule, can include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein.

By “cap structure” is meant chemical modifications which have been incorporated at either terminus of an oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples: the 5′-cap can be an inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By “abasic” or “abasic nucleotide” is meant sugar moieties lacking a base or having other chemical groups in place of a base at the 1′ position, (for more details see Wincott et al., International PCT publication No. WO 97/26270).

By “alkyl” group is meant a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) are preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) can be preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH. Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core or substrate binding regions of the enzymatic nucleic acid domain of an nucleic acid sensor molecule. Such modified bases can also be present at one or more positions within the sensor domain of the nucleic acid sensor molecule, for example to improve interaction with the target nucleic acid sequence.

By “unmodified nucleotide” is meant a nucleotide with one of the bases adenine, cytosine, guanine, thymine, uracil joined to the 1′ carbon of beta-D-ribo-furanose.

By “modified nucleotide” is meant a nucleotide that contains a modification in the chemical structure of an unmodified nucleotide base, sugar and/or phosphate.

By “Inozyme” or “NCH” motif is meant, an enzymatic nucleic acid molecule comprising an Inozyme or NCH motif as is generally described in Usman et al., U.S. patent application Ser. No. 09/877,526 herein incorporated by reference in its entirety, including the drawings.

By “G-cleaver” motif is meant, an enzymatic nucleic acid molecule comprising a motif as is generally described as G-cleaver in Usman et al., U.S. patent application Ser. No. 09/877,526, herein incorporated by reference in its entirety, including the drawings.

By “amberzyme” motif is meant, an enzymatic nucleic acid molecule comprising a motif as is generally described as amberzyme in Usman et al., US Patent application Ser. No. 09/877,526, herein incorporated by reference in its entirety, including the drawings.

By “zinzyme” motif is meant, an enzymatic nucleic acid molecule comprising a motif as is generally described as zinzyme in Usman et al., U.S. patent application Ser. No. 09/877,526, herein incorporated by reference in its entirety, including the drawings.

By “DNAzyme” is meant, an enzymatic nucleic acid molecule that does not require the presence of a 2′-OH group within it for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. Examples of DNAzymes are generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39; Perrin et al., 2001, JACS., 123, 1556. Additional DNAzyme motifs can be selected for using techniques similar to those described in these references, and hence, are within the scope of the present invention.

By “RNA” is meant a molecule comprising at least one ribonucleotide residue. By “ribonucleotide” or “2′—OH” is meant a nucleotide with a hydroxyl group at the 2′ position of a β-D-ribo-furanose moiety.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “system” is meant a group of substances or components that can be collectively combined or identified. A system can comprise a biological system, for example, an organism, cell, or components, extracts, and samples thereof. A system can further comprise an experimental or artificial system, where various substances or components are intentionally combined together. The “biological system” as used herein can be a eukaryotic system or a prokaryotic system, for example, a bacterial cell, plant cell or a mammalian cell, or of plant origin, mammalian origin, yeast origin, Drosophila origin, or archebacterial origin.

By “detectable response” is meant a chemical or physical property that can be measured, including, but not limited to, changes in temperature, pH, frequency, charge, capacitance, or changes in fluorescent, chromogenic, radioactive, enzymatic and/or chemiluminescent levels or properties that can then be detected using standard methods known in the art.

By “array” is meant an arrangement of entities in a pattern on a substrate, for example a two dimensional or three dimensional pattern (see for example Wagner et al., International PCT Publication Nos. WO 00/04390, WO 00/04389, and WO 00/04382). Non-limiting examples of substrates used as underlying or core material on which the array is generated include silicon, functionalized glass, germanium, PTFE (Polytetrafluoroethylene), polystyrene, gold, silver, or any other suitable material.

By “genome” is meant the complete set of genes found in or expressed in a particular system, such as a cell or organism, for example, a human cell or human.

By “genome map” is meant the functional relationship between different gene constituents of a genome.

By “genome scoring” is meant a process of identifying and measuring the presence of genes in a genome. Genome scoring can also refer to a system of ranking genes in terms of the relationship between a particular gene and a certain disease state or drug response in an organism, for example a human. Genome scoring can be used in determining the genotype of an organism.

By “disease specific genome” is meant a genome associated with a particular disease, disorder or condition.

By “treatment specific genome” is meant a genome associated with a particular treatment or therapy.

By “communication module” is meant a nucleic acid sequence or sequences that can link the sensor domain to the enzymatic nucleic acid domain of a nucleic acid sensor molecule.

The term “comprising” is meant to include, but is not limited to, whatever follows the word “comprising”. Thus, use of the term “comprising” indicates that the listed elements are required or mandatory, but that other elements are optional and may or may not be present. The term “consisting of” is meant including, and limited to, whatever follows the phrase “consisting of”. Thus, the phrase “consisting of” indicates that the listed elements are required or mandatory, and that no other elements can be present.

Other features and advantages of the invention will be apparent from the following description of the preferred embodiments thereof, and from the claims.

Detection of Nucleic Acids by Nucleic acid sensor molecules of the Invention

Nucleic Acid Sensor Molecules

In one embodiment, the invention features several approaches to detecting target nucleic acids, polynucleotides, and oligonucleotides in a system using nucleic acid sensor molecules (see for example FIG. 1). Activity of the nucleic acid sensor molecule is modulated via interaction of the sensor domain of the nucleic acid sensor molecule with the target nucleic acids, polynucleotides, and/or oligonucleotides. The nucleic acid sensors of the invention are designed such that the activity of the nucleic acid sensor is stimulated or as a direct consequence of the interaction between the target nucleic acid and the sensor domain of the nucleic acid sensor molecule.

A variety of different nucleic acid sensor molecule designs can be used to detect target signaling molecules in a system as described herein (see for example Usman et al., WO 01/66721, George et al., U.S. Pat. Nos. 5,834,186 and 5,741,679, Shih et al, U.S. Pat. No. 5,589,332, Nathan et al, U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842). In one embodiment, the present invention utilizes at least three components for proper function: nucleic acid sensor molecule, reporter molecule, and target nucleic acid, polynucleotide, and/or oligonucleotide. The nucleic acid sensor molecule is comprised of a sensor component and an enzymatic nucleic acid component connected by a communication module. The nucleic acid sensor molecule is, in a non-limiting example, in its inactive state when the sensor component is not interacting with a target nucleic acid, polynucleotide, and/or oligonucleotide. In the presence of a target nucleic acid, polynucleotide, and/or oligonucleotide, the sensor component interacts with the target nucleic acid sequence preferentially and causes the nucleic acid sensor molecule to be active. Alternately, in the presence of a target nucleic acid sequence (e.g., nucleic acid used for detection), the sensor component alters the overall structure of the enzymatic nucleic acid component, resulting in the nucleic acid sensor molecule becoming inactive.

When the sensor component is bound to the target nucleic acid molecule and the reporter molecule binds to the enzymatic nucleic acid component of the nucleic acid sensor molecule, a reaction can be catalyzed on the reporter molecule by the enzymatic nucleic acid component. For example, the reporter molecule can be cleaved or ligated. The cleavage or ligation event can then be detected by using a number of assays. For example, electrophoresis on a polyacrylamide gel would detect not only the full length reporter oligonucleotide but also any cleavage/ligation products that were created by the functional nucleic acid sensor molecule. The detection of these reaction products indicates the presence of the target nucleic acid molecule in the system. In addition, the reporter molecule can contain a fluorescent molecule at one end which fluorescence signal is quenched by another molecule attached at the other end of the reporter molecule. Cleavage of the reporter molecule in this case results in the disassociation of the florescent molecule and the quench molecule, resulting in a signal. Ligation of the reporter molecule would inversely result in attenuation of the florescent signal. This signal can be detected and/or quantified by methods known in the art (for example see Nathan et al., U.S. Pat. No. 5,871,914, Birkenmeyer, U.S. Pat. No. 5,427,930, and Lizardi et al., U.S. Pat. No. 5,652,107, George et al., U.S. Pat. Nos. 5,834,186 and 5,741,679, and Shih et al., U.S. Pat. No. 5,589,332).

Detection Formats

The methods of the instant invention can be practiced in a variety of different detection formats. In one embodiment, the invention is practiced using an arrayed format in which a series of nucleic acid sensor molecules specific for different nucleic acid molecules is used to detect one or more different target nucleic acid molecules in a system. Such arrays provide the capability to screen many different targets in a parallel multiplexed format. For example, an array of the invention can comprise nucleic acid sensor molecules specific for a disease-specific gene, a cellular genome, or an entire human genome. In another embodiment, the array of the invention is in a multi-well and/or multi-channel plate format, wherein each well comprises a particular nucleic acid sensor molecule of the invention (see for example FIG. 7). In yet another embodiment, the array of the invention is arranged on a surface or chip format comprising a solid substrate, such as a silicon based chip or bead, wherein discrete regions of the surface correspond to particular nucleic acid sensor molecules or particular groups of nucleic acid sensor molecules of the invention. Nucleic acid sensor molecules of the invention can be covalently attached to the surface of the array, for example, to an organic or inorganic thin film coating on the surface of the substrate (see for example Wagner et al., International PCT Publication Nos. WO 00/04390, WO 00/04389, and WO 00/04382).

Typically, each discrete region or the array comprises one nucleic acid sensor molecule of the invention. The arrays of the present invention can comprise any number of nucleic acid sensor molecules. For example, an array can comprise 1-100, 1-1000, or 1-10,000 nucleic acid sensor molecules, or greater than 1×10⁴, 1×10⁵, or 1×10⁶ nucleic acid sensor molecules.

Nucleic Acid Molecule Synthesis

The nucleic acid molecules of the invention, including certain nucleic acid sensor molecules can be synthesized using the methods described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59. Such methods make use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table 1 outlines the amounts and the contact times of the reagents used in the synthesis cycle. Alternatively, syntheses at the 0.2 μmol scale can be done on a 96-well plate synthesizer, such as the PG2100 instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Cleavage from the solid support and deprotection of the oligonucleotide is typically performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃. An alternative deprotection cocktail for use in the one pot protocol comprises the use of aqueous methylamine (0.5 ml) at 65° C. for 15 min followed by DMSO (0.8 ml) and TEA.3HF (0.3 ml) at 65° C. for 15 min. A similar methodology can be employed with 96-well plate synthesis formats by using a Robbins Scientific Flex Chem block, in which the reagents are added for cleavage and deprotection of the oligonucleotide.

For anion exchange desalting of the deprotected oligomer, the TEAB solution is loaded onto a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile. Alternatively, for oligonucleotides synthesized in a 96-well format, the crude trityl-on oligonucleotide is purified using a 96-well solid phase extraction block packed with C18 material, on a Bahdan Automation workstation.

The average stepwise coupling yields are typically >98% (Wincott et al., 1995 Nucleic Acids Res. 23, 2677-2684). Those of ordinary skill in the art will recognize that the scale of synthesis can be adapted as larger or smaller than the example described above including but not limited to 96 well format, all that is important is the ratio of chemicals used in the reaction.

To ensure the quality of synthesis of nucleic acid molecules of the invention, quality control measures are utilized for the analysis of nucleic acid material. Capillary Gel Electrophoresis, for example using a Beckman MDQ CGE instrument, can be ulitized for rapid analysis of nucleic acid molecules, by introducing sample on the short end of the capillary. In addition, mass spectrometry, for example using a PE Biosystems Voyager-DE MALDI instrument, in combination with the Bohdan workstation, can be utilized in the analysis of oligonucleotides, including oligonucleotides synthesized in the 96-well format.

The nucleic acids of the invention can also be synthesized in two parts and annealed to reconstruct the nucleic acid sensor molecules (Chowrira and Burke, 1992 Nucleic Acids Res., 20, 2835-2840). The nucleic acids are also synthesized enzymatically using a variety of methods known in the art, for example as described in Havlina, International PCT publication No. WO 9967413, or from DNA templates using bacteriophage T7 RNA polymerase (Milligan and Uhlenbeck, 1989, Methods Enzymol. 180, 51). Other methods of enzymatic synthesis of the nucleic acid molecules of the invention are generally described in Kim et al., 1995, Biotechniques, 18, 992; Hoffman et al., 1994, Biotechniques, 17, 372; Cazenare et al., 1994, PNAS USA, 91, 6972; Hyman, U.S. Pat. No. 5,436,143; and Karpeisky et al., International PCT publication No. WO 98/28317)

Alternatively, the nucleic acid molecules of the present invention can be synthesized separately and joined together post-synthetically, for example by ligation (Moore et al, 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are preferably modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid sensor molecules are purified by gel electrophoresis using known methods or are purified by high pressure liquid chromatography (HPLC; See Wincott et al., Supra, the totality of which is hereby incorporated herein by reference) and are re-suspended in water.

Optimizing Nucleic Acid Molecule Activity

Synthesizing nucleic acid molecules with modifications (base, sugar and/or phosphate) that prevent their degradation by serum ribonucleases can increase their potency (see e.g., Eckstein et al., International Publication No. WO92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. Chemical modification can also be used to modulate the binding activity of nucleic acid molecules of the invention. Modifications which enhance their efficacy, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance activity by modification with various groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into nucleic acid sensor molecule molecules without inhibiting catalysis. In view of such teachings, similar modifications can be used as described herein to modify the nucleic acid molecules of the instant invention.

Nucleic acid molecules having chemical modifications which maintain or enhance activity are provided. Such nucleic acid is also generally more resistant to nucleases than unmodified nucleic acid. Thus, in the presence of biological fluids, or in cells, the activity can not be significantly lowered. Clearly, nucleic acid molecules must be resistant to nucleases in order to function as effective diagnostic agents, whether utilized in vitro and/or in vivo. Improvements in the synthesis of RNA and DNA (Wincott et al., 1995 Nucleic Acids Res. 23, 2677; Caruthers et al., 1992, Methods in Enzymology 211,3-19; Karpeisky et al., International PCT publication No. WO 98/28317) (incorporated by reference herein) have expanded the ability to modify nucleic acid molecules by introducing nucleotide modifications to enhance their nuclease stability as described above.

In another aspect the nucleic acid molecules comprise a 5′ and/or a 3′-cap structure.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Karpeisky et al., WO 98/28317, respectively, which are both incorporated by reference herein in their entireties.

In one embodiment, nucleic acid molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets.

### EXAMPLES

The following are non-limiting examples showing techniques of the inventive method for isolation of the nucleic acid molecules of the invention.

### Example 1

### Engineering RNA Molecular Switches that Respond to Oligonucleotides

Applicant has implemented methodology for generating oligonucleotide-sensitive nucleic acid molecules by conducting in vitro selection using a partially randomized RNA population based on the hammerhead self-cleaving ribozyme (FIG. 1A). Other generalized nucleic acid sensor motifs that are contemplated by the instant invention are shown in FIG. 7. The RNA construct used to express the population was designed to take advantage of the fact that the hammerhead ribozyme activity is sensitive to the structure of stem II. In this construct, stem II is replaced with two random-sequence domains that are separated by a domain of defined nucleotide sequence (sensor domain). It was expected that the vast majority of RNAs in the population would be able to bind the target oligonucleotide via the sensor domain. In many cases this binding even is expected to control the formation of stem II, which would allow the modulation of nucleic acid sensor activity.

Isolation of Oligonucleotide-Sensitive Ribozymes

The oligonucleotide-responsive nucleic acid sensors begin to dominate the population of selected nucleic acids very early in the process (FIG. 1B), and very efficient nucleic acid sensors form the bulk of the population after 11 generations. Based on kinetic analysis of the population, we believe that many ribozymes function with near wild-type catalytic activity. FIG. 1 shows the in vitro selection of oligonucleotide-sensitive nucleic acid sensor molecules. FIG. 1A shows the construct used to initiate the in vitro selection process. The hammerhead catalytic platform (including stems I, II and III) is modified to carry two 20-nucleotide random-sequence domains in place of stem II, which are separated by a 10-nucleotide domain of defined sequence (sensor domain). Arrowhead identifies the site of ribozyme self-cleavage. FIG. 1B shows a plot depicting the ratio of ribozyme activity in the presence (+) vs. the absence (−) of an 18-nucleotide effector molecule. Incubation time in the positive selection (+oligomer) was carried out as denoted at the top of the plot (15 min, 5 min, and 1 min). Negative selection was carried out for 5 hrs (a) or for five consecutive 30-minute intervals that were punctuated by alkaline denaturation (b). The asterisk denotes the rounds of selection wherein the order of reagent addition changed from (RNA; buffer; water; oligonucleotide; Mg2+) to (RNA; buffer; water; Mg2+; oligonucleotide). The oligonucleotide sequence (effector molecule) 5′ACTGCCATGGAGGAGCCG (SEQ ID NO: 18) is complementary to the fixed oligomer-binding domain (sensor domain) of the population between nucleotides 5 and 14.

The population at G11 was cloned and a preliminary sequence analysis and an in vitro assay was conducted with about a dozen individuals. The sequences of the allosteric domains of these representative clones are depicted in FIG. 2. It is clear from these clones that G11 is populated with a diverse collection of nucleic acid variants, which indicates that there are many sequence classes that can achieve selective modulation of ribozyme action. FIG. 2 shows a sequence analysis of the of 16 nucleic acid sensor molecules (5′ to 3′) that respond to specific oligonucleotides. The first G and the last C comprise the first base pair of stem II in the original nucleic acid catalyst (see FIG. 1A). Bold sequences represent the original fixed sequence sensor domain of the construct. Clone 1 has experienced a single mutation within this domain. Underlined nucleotides identify nucleotides of the random-sequence domains that are complementary to the effector (target sequence). All clones except 7, 10 and 94 carry a common sequence element (5′-CGAAACG) that appears to serve a critical role in nucleic acid switching.

Selection Protocol for Isolation of Oligonucleotide-dependent Hammerhead Ribozymes Negative Selection

The starting RNA population was comprised of greater than 10¹² sequence variants. The RNA population was combined (final concentration=10 μM) with reaction buffer (50 mM Tris-HCl [pH 7.5 at 23° C.]; 20 mM MgCl2) and incubated at 23° C. for 15 hr and the reaction products separated by denaturing (7 M urea) 10% polyacrylamide gel electrophoresis (PAGE). The uncleaved RNA was isolated by excising the precursor (uncleaved RNA) band and the RNA was recovered by a standard crush/soak method. The resulting RNA was precipitated using ethanol and the dried pellet resuspended in deionized water (dH2O).

Positive Selection

The negative-selected RNA population was combined (final concentration=10 μM) with reaction buffer (50 mM Tris-HCl [pH 7.5 at 23° C.]; 20 mM MgCl2). The DNA effector molecule was then added (final concentration of 1 μM) to initiate the reaction comprising incubation at 23° C. for 15 min and the reaction products separated by denaturing PAGE. The cleaved RNA was isolated by excising the appropriate cleavage product band and recovering the RNA by a standard crush/soak method. The resulting RNA was precipitated using ethanol and the dried pellet resuspended in deionized water (dH2O).

Amplification

Reverse transcription and polymerase chain reaction (RT-PCR) protocols were conducted according to standard methods. The resulting double-stranded DNA was used as template for in vitro transcription with T7 RNA polymerase under standard reaction conditions.

Protocol Variations

Various parameters of the protocol can be altered to apply selective pressure on specific characteristics of the nucleic acid sensor molecules. For example, decreasing incubation time during positive selection will favor the isolation of nucleic acid sensor molecules with higher rate constants when bound to the effector. Increasing incubation time during negative selection will favor the isolation of nucleic acid sensor molecules that have lower rate constants for nucleic acid sensor molecule cleavage in the absence of effector. Lowering the effector concentration will favor the isolation of nucleic acid sensor molecules with improved affinity for the effector.

In the current example, early rounds of selection used 15 minute incubation for the positive selection reaction. This was progressively reduced to favor the isolation of higher-speed nucleic acid sensor molecules. Also, early rounds of selection made use of a separate reaction buffer wherein Tris was added first, effector was added next, and Mg2+ was added last. This protocol gave rise to a population of nucleic acid sensor molecules that largely required this order of addition (nucleic acid sensor molecules could not become active if Tris and Mg2+ were added in combination, followed by addition of effector). In later rounds, the order of addition was altered as outlined above, and this change permitted the selection of nucleic acid sensor molecules that are able to switch from the OFF state to the ON state when effector is applied.

An essential component of the selection process is the use of modified negative selection protocols that disfavor the isolation of selfish molecules. For example, in later rounds, we employed negative selection reactions that were comprised of repetitive cycles (3 to 5) of ˜1 hr incubation at 23° C. followed by a 30 sec incubation at 90° C. This is expected to permit misfolded RNAs to become denatured and refolded in order to maximize the removal of nucleic acid sensor molecules that do not require effector to cleave.

Functional Characteristics of Individual Ribozymes

Assays of 14 clones were conducted to make a preliminary assessment of their allosteric activity and specificity. These clones remain largely inactive when incubated in the absence of matched effector (target nucleic acid molecule) and when incubated in the presence of a non-complementary effector (FIG. 3). However, each exhibits significant levels of activity when the target oligonucleotide is included in the reaction mixture. Certain clones (1 and 3) appear to be selective for DNA as the effector, while others are activated regardless of whether the target oligonucleotide is DNA or RNA. FIG. 3 shows an activity assay for fourteen oligonucleotide-sensitive nucleic acid sensor molecules. Clones were incubated under in vitro selection conditions for the time indicated in the absence of target (lane 5) or in the presence of various target oligonucleotides as indicated. DNA-1 is the matched DNA molecule, DNA-0 is a non-complementary DNA oligomer, and RNA-1 is the RNA version of DNA-1. Reactions were incubated in 50 mM Tris-HCl (pH 7.5 at 23° C.), 20 mM MgCl2, ˜0.1 pmole ribozyme and 1 pmole of each target oligonucleotide as indicated.

The utility of oligonucleotide-sensitive nucleic sensor acid molecules as biosensor elements is dictated in part by the selectivity that a nucleic acid sensor exhibits. The goal is to identify nucleic acid sensors with two main features: 1) universal target specificity, and 2) high discrimination between matched and mismatched target sequences. A “universal” nucleic acid sensor molecule will be one that can be engineered to selectively perform with nearly any defined effector sequence, thus permitting the rapid creation of oligonucleotide-sensitive nucleic acid sensor molecules with unique effector specificities.

Target Discrimination by Representative Nucleic Acid Sensor Molecules

The level and type of discrimination exhibited by clone 40 was examined by testing a series of closely related mutants of the DNA 1 sequence (FIG. 4A). The sensor molecule is capable of distinguishing between the matched DNA 1 sequence and the completely unrelated DNA 0 sequence, as determined by the catalytic activity of the nucleic acid sensor (FIG. 4B). However, mutant effectors with sequences that are closely related to DNA 1 permit varying levels of sensor activity that are entirely consistent with a thermodynamic basis for effector discrimination. Specifically, there is a correlation between the loss of binding stability (indicated by high ΔΔG values) and the reduction in the fraction of precursor RNA cleaved (FIG. 4C). Oligonucleotide detection systems (e.g. gene chips) should provide a similar level of discrimination between closely related target sequences. FIG. 4 above shows an analysis of the effector specificity of clone 40. (A) Sequence of the original effector DNA (DNA 1), the sequences of eight mutants (1-8), and the sequence of the mismatched DNA oligonucleotide (DNA 0). (B) Gel analysis of a 5′ 32P-labeled sensor after incubation under in vitro selection conditions for the times indicated in the absence of effector or in the presence of various target oligonucleotides as indicated. Reactions were incubated as indicated in the legend to FIG. 3. (C) Top: Comparison of the differences in the free energy of duplex formation between the DNA 1 complex with sensor versus the free energy of duplex formation between the various mutant DNAs and the sensor. Bottom: Fraction of sensor precursor cleaved in the presence of each effector DNA as indicated.

Similarly, the characteristics of clone 11 were also examined by testing a series of mutant effector molecules (FIG. 5A). As with clone 40, the sensor is capable of distinguishing between the DNA 1 and DNA 0 molecules (FIG. 5B). Likewise, mutant effectors with sequences that are closely related to DNA 1 permit varying levels of ribozyme activity that are largely consistent with a thermodynamic basis for effector discrimination (FIG. 5C). However, the clone 11 sensor responds uniquely to at least three mutant effectors (FIG. 5C, asterisks), indicating that effector recognition is guided in part by thermodynamic principles and in part by kinetic mechanisms. This complexity could be exploited to create sensors that can exceed the target selectivity that is normally observed with existing oligonucleotide detection systems. FIG. 5 above Preliminary analysis of the effector specificity of clone 11. (A) See the legend to FIG. 4A. (B) See the legend to FIG. 4B. (C) See the legend to FIG. 4C. The asterisks denote mutants that bring about ribozyme action that is not consistent with a mechanism that involves purely thermodynamic base pairing considerations. Specifically, the activity of the ribozyme is lower than expected when mutant 1 is used, and is higher than expected when mutants 3 and 8 are used. It is important to note however, that these are preliminary results and that the findings need further confirmation.

Structure and Mechanism of Allosteric Modulation of Ribozyme Function

RNA secondary structure prediction algorithms were used to construct models of the “On” and “Off” states of each class of RNA switches. For example, the most stable structure predicted for class 7 in the absence of oligonucleotide effector has appropriately-formed stem I and stem III elements of the hammerhead ribozyme, but stem II of the hammerhead ribozyme is disrupted by competing base pairing arrangements (FIG. 6A). In contrast, the most stable structure predicted for the complex between the RNA switch and the oligonucleotide effector allows for the formation of an imperfect but expanded version of stem II, which presumably is consistent with sensor function (FIG. 6B). FIG. 6 shows models for the inactive (A) and active (B) states of the clone 7 molecular switch. Note that clone 7 has acquired additional base complementation with the oligonucleotide target relative to the original 10 nucleotides that were fixed in the original construct.

Other Uses

The nucleic acid sensor molecules of this invention can be used as diagnostic tools to examine genetic drift and mutations within diseased cells or to detect the presence of specific nucleic acids in a cell. In addition, the nucleic acid sensor molecules of this invention can be used as therapeutic tools. The close relationship between nucleic acid sensor molecule activity and the structure of the target nucleic acid, polynucleotide, or oligonucleotide allows the detection of mutations in any region of the molecule that alters the activity and three-dimensional structure of the target nucleic acid. By using multiple nucleic acid sensor molecules described in this invention, one can determine nucleotide(s) which are important to nucleic acid structure and function in vitro, as well as in cells and tissues. Furthermore, inhibition of target gene expression/function with nucleic acid sensor molecules can be used in therapeutic applications to inhibit gene expression or gene function and define the role (essentially) of specified gene products, e.g., proteins, in the progression of disease. Using this technique, other genetic targets can be defined as important mediators of the disease. The molecules, reagents, and methods described herein can be used to improve diagnosis of disease-state and develop improved treatment of disease progression by affording the possibility of combinational therapies (e.g., multiple nucleic acid sensor molecules targeted to different genes, nucleic acid target molecules coupled with known small molecule inhibitors, or intermittent treatment with combinations of nucleic acid sensor molecules and/or other chemical or biological molecules).

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE 1

| | | | | | |
|---|---|---|---|---|---|
| | | | | Wait | |
| | | | Wait | Time* | Wait |
| | Equiva- | | Time* | 2′-O- | Time* |
| Reagent | lents | Amount | DNA | methyl | RNA |

A. 2.5 μmol Synthesis Cycle ABI 394 Instrument

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl | 23.8 | 238 | μL | 45 | sec | 2.5 | min | 7.5 | min |
| Tetrazole | | | | | | | | | |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl | 38.7 | 31 | μl | 45 | sec | 233 | min | 465 | sec |
| Tetrazole | | | | | | | | | |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

| | | | | | |
|---|---|---|---|---|---|
| | Equiva- | | | | |
| | lents: | | | Wait | |
| | DNA/2′- | Amount: | Wait | Time* | Wait |
| | O-methyl/ | DNA/2′-O- | Time* | 2′-O- | Time* |
| Reagent | Ribo | methyl/Ribo | DNA | methyl | Ribo |

C. 0.2 μmol Synthesis Cycle 96 well Instrument

| | | | | | | | | | |
|---|---|---|---|---|---|---|---|---|---|
| Phosphoramidites | 22/33/66 | 40/60/120 | μL | 60 | sec | 180 | sec | 360 | sec |
| S-Ethyl | 70/105/210 | 40/60/120 | μL | 60 | sec | 180 | min | 360 | sec |
| Tetrazole | | | | | | | | | |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

We claim:

1. A method comprising:
(a) generating a mixture of nucleic acid sequences comprising a first domain having sequence complementary to a target nucleic acid molecule wherein the first domain is flanked on either side by a second and third domain each having random sequence and wherein each of the second and third domains is linked to a portion of a fourth domain comprising an enzymatic nucleic acid sequence;
(b) contacting the mixture of nucleic acid sequences from (a) with the target nucleic acid molecule under conditions suitable for the target nucleic acid molecule to interact with the first domain and activate the fourth domain and catalyze a chemical reaction on a reporter molecule;
(c) partitioning any active nucleic acid sequences from the rest of the mixture;
(d) amplifying the active nucleic acid sequences to yield an enriched mixture of active nucleic acid sequences; and
(e) repeating steps (b)-(d), as necessary, to identify a nucleic acid sensor molecule capable of catalyzing a chemical reaction in the presence of the target nucleic acid molecule.

2. The method of claim 1, wherein the first domain of (a) is from about 4 to about 100 nucleotides in length.

3. The method of claim 1, wherein the first domain of (a) is from about 8 to about 50 nucleotides in length.

4. The method of claim 1, wherein the first domain of (a) is from about 10 to about 20 nucleotides in length.

5. The method of claim 1, wherein the second and third domains of (a) can be of equal or unequal length.

6. The method of claim 1, wherein the second and third domains of (a) are independently from about 4 to about 200 nucleotides in length.

7. The method of claim 1, wherein the second and third domains of (a) are independently from about 10 to about 50 nucleotides in length.

8. The method of any of claims 1-4, wherein the fourth domain of (a) comprises a hammerhead, inozyme, g-cleaver, hairpin, Zinzyme, Amberzyme, or DNAzyme enzymatic nucleic acid sequence.


SEQUENCE LISTING (28 sequences)

SEQ ID NO: 1 - RNA, 52 nt, Artificial Sequence
  Description of Artificial Sequence: Nucleic Acid Sensor Domain
guauggcaguugaaacggcauuccuccacggcaccagcuacaggauuuagac

SEQ ID NO: 2 - RNA, 52 nt, Artificial Sequence
  Description of Artificial Sequence: Nucleic Acid Sensor Domain
gugggcaguugaaacgguuauuccuccaugguccccucggacugccgucguc

SEQ ID NO: 3 - RNA, 52 nt, Artificial Sequence
  Description of Artificial Sequence: Nucleic Acid Sensor Domain
gugaaggcgaaacggaacccuuccuccauggugugcucaagcuuauuguguc

SEQ ID NO: 4 - RNA, 53 nt, Artificial Sequence
  Description of Artificial Sequence: Nucleic Acid Sensor Domain
gauuaaacgaaacgucugcuucuccuccauggccgacggagucaacgcugccc

SEQ ID NO: 5 - RNA, 52 nt, Artificial Sequence
  Description of Artificial Sequence: Nucleic Acid Sensor Domain
guuuuccuuacucaaaccagcuccuccauggcaguuauacaugaaggaucac

SEQ ID NO: 6 - RNA, 52 nt, Artificial Sequence
  Description of Artificial Sequence: Nucleic Acid Sensor Domain
gucacagacgcuuacccuguguccuccauggcagucuaaugaagaucggcac

SEQ ID NO: 7 - RNA, 52 nt, Artificial Sequence
  Description of Artificial Sequence: Nucleic Acid Sensor Domain
guuggcaguugaaacgguuacuccuccaugguggacuucgcaccaccauugc

SEQ ID NO: 8 - RNA, 52 nt, Artificial Sequence
  Description of Artificial Sequence: Nucleic Acid Sensor Domain
gcaguaagcgaaacgaaccgguccuccauggcaguuacucugagggaccaac

SEQ ID NO: 9 - RNA, 53 nt, Artificial Sequence
  Description of Artificial Sequence: Nucleic Acid Sensor Domain
guucaacgaaacggauccgucuccuccauggcuguugauugaguaacgucccc

SEQ ID NO: 10 - RNA, 52 nt, Artificial Sequence
  Description of Artificial Sequence: Nucleic Acid Sensor Domain
gcuuccaagcagaaacggggcuccuccauggcaccgugaauguagccuaccc

SEQ ID NO: 11 - RNA, 51 nt, Artificial Sequence
  Description of Artificial Sequence: Nucleic Acid Sensor Domain
guacuugcgaaacggaauucuuccuccauggcagcgaccggaagaaacacc

SEQ ID NO: 12 - RNA, 52 nt, Artificial Sequence
  Description of Artificial Sequence: Nucleic Acid Sensor Domain
gcaaaacgcgaaacgcucggcuccuccauggugcgagggcaucucuaccgcc

SEQ ID NO: 13 - RNA, 52 nt, Artificial Sequence
  Description of Artificial Sequence: Nucleic Acid Sensor Domain
gucauugacgaaacgcucggcuccuccauggcgcagucaacccucuuguucc

SEQ ID NO: 14 - RNA, 52 nt, Artificial Sequence
  Description of Artificial Sequence: Nucleic Acid Sensor Domain
guuaaacgaaacgcacucggcuccuccauggaaggaaguucuuaugucuacc

SEQ ID NO: 15 - RNA, 52 nt, Artificial Sequence
  Description of Artificial Sequence: Nucleic Acid Sensor Domain
gcucgcucuuccuuaaaucgcuccuccauggcgcaccaaucugaagcgcgac

SEQ ID NO: 16 - RNA, 52 nt, Artificial Sequence
  Description of Artificial Sequence: Nucleic Acid Sensor Domain
ggaugaagcgaaacggaacgcuccuccaugguagacgcccucucaaaauacc

SEQ ID NO: 17 - DNA, 18 bp, Artificial Sequence
  Description of Artificial Sequence: Nucleic Acid Sensor Domain
gccgaggaggtaccgtca

SEQ ID NO: 18 - DNA, 18 bp, Artificial Sequence
  Description of Artificial Sequence: Nuceic Acid Effector Molecule
actgccatggaggagccg

SEQ ID NO: 19 - RNA, 10 nt, Artificial Sequence
  Description of Artificial Sequence: Nucleic Acid Sensor Domain
uccuccaugg

SEQ ID NO: 20 - RNA, 38 nt, Artificial Sequence
  Description of Artificial Sequence: Portion of Nucleic Acid Sensor Molecule
ggauaauagccguagguugcgaaagcgacccugaugag

SEQ ID NO: 21 - RNA, 20 nt, Artificial Sequence
  Description of Artificial Sequence: Portion of Nucleic Acid Sensor Molecule
cgaaacgguagcgagagcuc

SEQ ID NO: 22 - DNA, 18 bp, Artificial Sequence
  Description of Artificial Sequence: Nucleic Acid Sensor Molecule
actgccatggaggagccg

SEQ ID NO: 23 - DNA, 15 bp, Artificial Sequence
  Description of Artificial Sequence: Nucleic Acid Effector Molecule
gtgccaacgttaccg

SEQ ID NO: 24-25 - skipped (2 entries)

SEQ ID NO: 26 - RNA, 108 nt, Artificial Sequence
  Description of Artificial Sequence: Nucleic Acid Sensor Molecule
ggauaauagccguagguugcgaaagcgacccugaugaguuuuccuuacucaaaccagcuccuccauggcaguuauacaugaaggaucacgaaacgguagcgagagcuc

SEQ ID NO: 27 - DNA, 18 bp, Artificial Sequence
  Description of Artificial Sequence: Nucleic Acid Effector Molecule
actgccatggaggagccg

SEQ ID NO: 28 - RNA, 108 nt, Artificial Sequence; identical to SEQ ID NO: 26
  Description of Artificial Sequence: Nucleic Acid Sensor Molecule